(19) United States
(12) Patent Application Publication
(10) Pub. No.: US 2015/0118737 A1
(43) Pub. Date: Apr. 30, 2015

(54) CELL CULTURE DEVICE

(71) Applicant: SAMSUNG ELECTRO-MECHANICS CO., LTD., Suwon (KR)

(72) Inventors: Sang Jin KIM, Suwon (KR); Bo Sung KU, Suwon (KR)

(73) Assignee: SAMSUNG ELECTRO-MECHANICS CO., LTD., Suwon (KR)

(21) Appl. No.: 14/147,387

(22) Filed: Jan. 3, 2014

(30) Foreign Application Priority Data
Oct. 28, 2013 (KR) 10-2013-0128353

(51) Int. Cl. G01N 33/50

(52) U.S. Cl. CPC G01N 33/5011
USPC 435/287.1; 435/289.1; 435/297.2

(57) ABSTRACT

There is provided a cell culture device including: a drug storing chamber; a cell culture chamber connected to the drug storing chamber and disposed in a position higher than that of the drug storing chamber; a pump unit circulating a drug from the drug storing chamber to the cell culture chamber; and an opening and closing unit closing an opened part of the drug storing chamber so that the drug storing chamber is isolated from ambient atmosphere.

### CROSS-REFERENCE TO RELATED APPLICATION

This application claims the benefit of Korean Patent Application No. 10-2013-0128353 filed on Oct. 28, 2013, with the Korean Intellectual Property Office, the disclosure of which is incorporated herein by reference.

### BACKGROUND

The present disclosure relates to a cell culture device.

Demand for a biomedical devices and biotechnology for rapidly diagnosing various human diseases has recently increased. Therefore, development of an devices and tools for experiments, capable of rapidly providing diagnostic results specific diseases, testing for which previously required extended periods of time in hospitals or laboratories, has been actively undertaken.

Meanwhile, in order to develop new medicines and determine the stability thereof, it is necessary to observe reactions between new medicines (that is, drugs) and cells. Generally, experiments related to the reaction between drugs and cells are performed using a culture dish, or the like.

However, since a reaction between the drug and the cells made in the Petri dish is very different from a reaction between the drug and the cells made in a human body, it is difficult to accurately observe or inspect the reaction between the drug and the cells only with a result of the experiment using the Petri dish. Therefore, there is a need to develop a culture device capable of circulating a drug medium in order to observe a reaction between the drug and cells in an environment similar to that of a human body.

The related art for circulating a drug has been disclosed in Patent Documents 1 and 2.

### RELATED ART DOCUMENT

(Patent Document 1) JP1993-336945 A
(Patent Document 2) JP1993-276922 A

### SUMMARY

An aspect of the present disclosure may provide a cell culture device capable of constantly and accurately circulating a small amount of a drug.

According to an aspect of the present disclosure, a cell culture device may include: a drug storing chamber; a cell culture chamber connected to the drug storing chamber and disposed in a position higher than that of the drug storing chamber; a pump unit circulating a drug from the drug storing chamber to the cell culture chamber; and an opening and closing unit closing an opened part of the drug storing chamber so that the drug storing chamber is isolated from ambient atmosphere.

The cell culture device may further include a flow rate measuring unit measuring a flow rate of the drug circulated from the drug storing chamber to the cell culture chamber.

The cell culture device may further include a control unit comparing a measured signal received from the flow rate measuring unit with a preset value and controlling an operation of the opening and closing unit.

The control unit may include a logic circuit operating the opening and closing unit so as to open the opened part when the measured signal is equal to the preset value or less and operating the opening and closing unit so as to close the opened part when the measured signal exceeds the preset value.

The pump unit may be disposed below the drug storing chamber.

The drug storing chamber may be provided in plural.

The cell culture chamber may be provided in plural.

The cell culture chamber may include at least one channel.

The at least one channel may have a zigzag form.

The cell culture device may further include a filtering unit filtering foreign objects from the drug circulated from the drug storing chamber to the cell culture chamber.

According to another aspect of the present disclosure, a cell culture device may include: a housing having a drug storing chamber and a cell culture chamber formed therein; a pump unit circulating a drug from the drug storing chamber to the cell culture chamber; and an opening and closing unit closing an opened part of the drug storing chamber so that the drug storing chamber is isolated from ambient atmosphere.

The cell culture device may further include a flow rate measuring unit measuring a flow rate of the drug circulated from the drug storing chamber to the cell culture chamber.

The cell culture device may further include a control unit comparing a measured signal received from the flow rate measuring unit with a preset value and controlling an operation of the opening and closing unit.

The control unit may include a logic circuit operating the opening and closing unit so as to open the opened part when the measured signal is equal to the preset value or less and operating the opening and closing unit so as to close the opened part when the measured signal exceeds the preset value.

The pump unit may be disposed below the drug storing chamber.

The drug storing chamber may be provided in plural.

The cell culture chamber is provided in plural.

The cell culture chamber may include at least one channel.

The at least one channel may have a zigzag form.

The cell culture device may further include a filtering unit filtering foreign objects from the drug circulated from the drug storing chamber to the cell culture chamber.

The cell culture device may further include a humidity controlling unit formed in the housing so as to constantly maintain humidity within the cell culture chamber.

The humidity controlling unit may include: a water storing chamber formed in the housing; and a heater evaporating water stored in the water storing chamber.

The cell culture device may further include a cell chip mounted on the cell culture chamber.

The cell chip may include a plurality of protrusions extended into the cell culture chamber and having cell materials fixed thereto, respectively.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a view illustrating a schematic configuration of a cell culture device according to an exemplary embodiment of the present disclosure;

FIG. 2 is a view illustrating another form of the cell culture device shown in FIG. 1;

FIGS. 3 and 4 are views illustrating an operating state of the cell culture device shown in FIG. 2;

FIG. 5 is a view illustrating another form of the cell culture device shown in FIG. 1;

FIG. 6 is a plan view illustrating another form of the cell culture device shown in FIG. 1;

FIG. 7 is a view illustrating another form of the cell culture device shown in FIG. 1;

FIGS. 8 and 9 are views illustrating another form of a cell culture chamber shown in FIG. 1;

FIG. 10 is a perspective view of a cell culture device according to another exemplary embodiment of the present disclosure;

FIG. 11 is a cross-sectional view of the cell culture device taken along line A-A of FIG. 10;

FIG. 12 is a perspective view illustrating another form of the cell culture device shown in FIG. 10; and

FIGS. 13 and 14 are cross-sectional views illustrating another form of the cell culture device taken along line A-A of FIG. 10.

### DETAILED DESCRIPTION

Hereinafter, embodiments of the present invention will be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions of elements may be exaggerated for clarity, and the same reference numerals will be used throughout to designate the same or like elements.

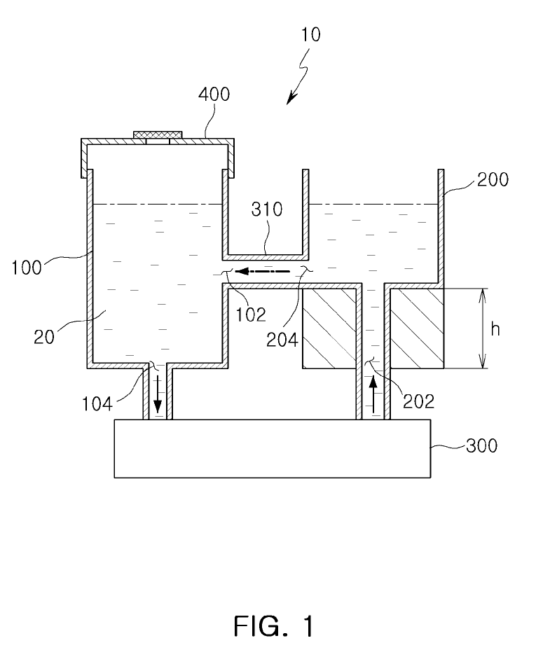

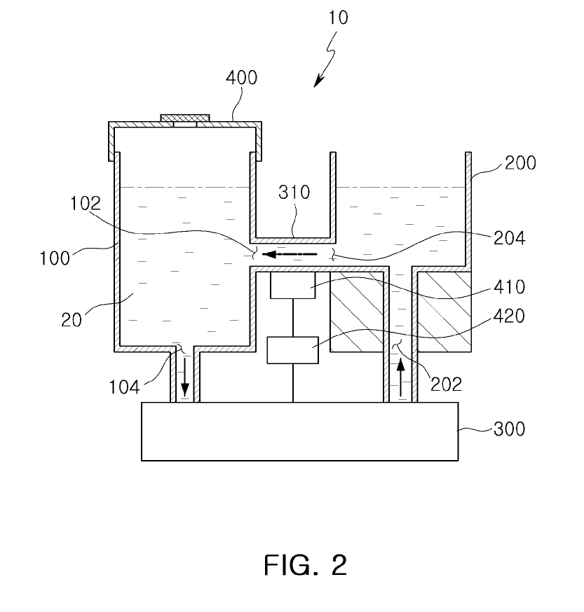

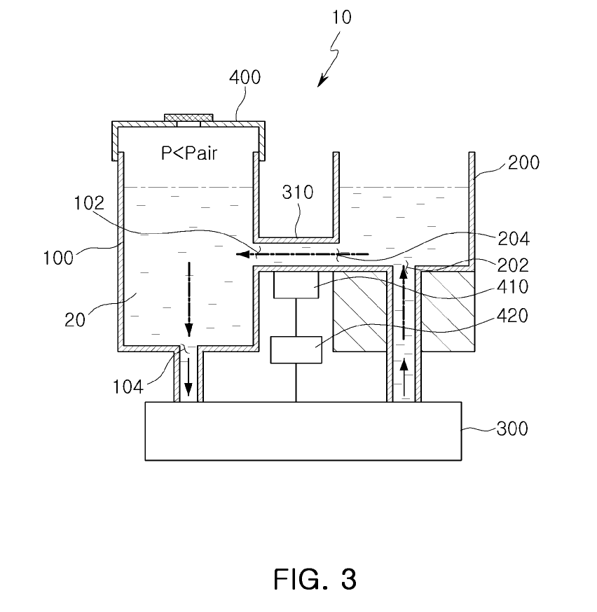

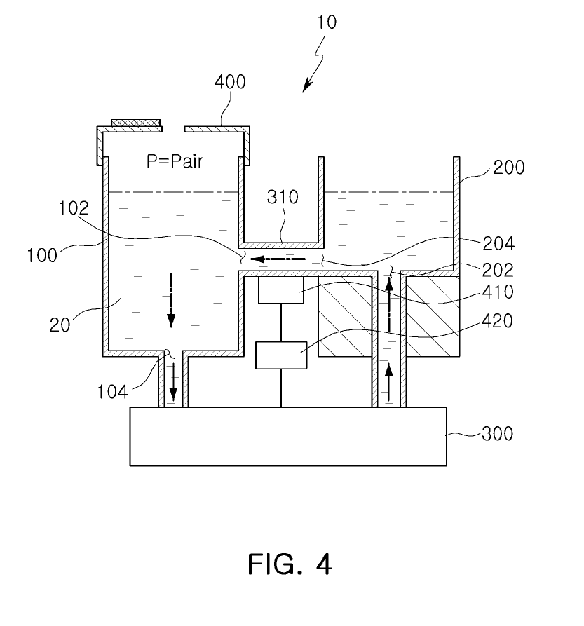

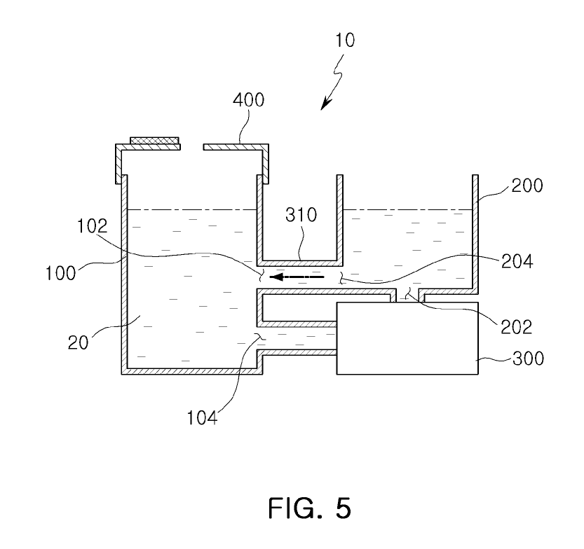

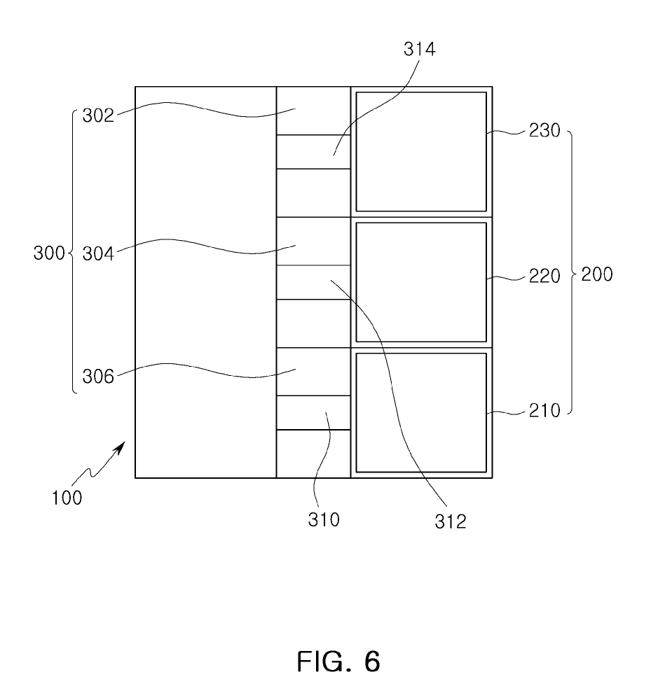

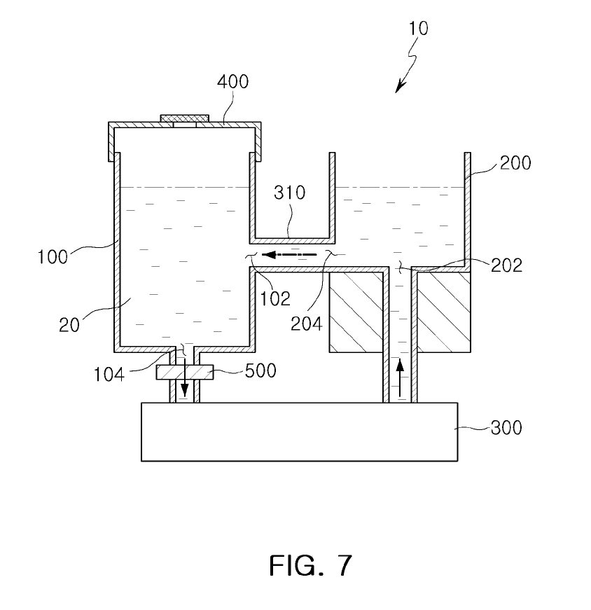

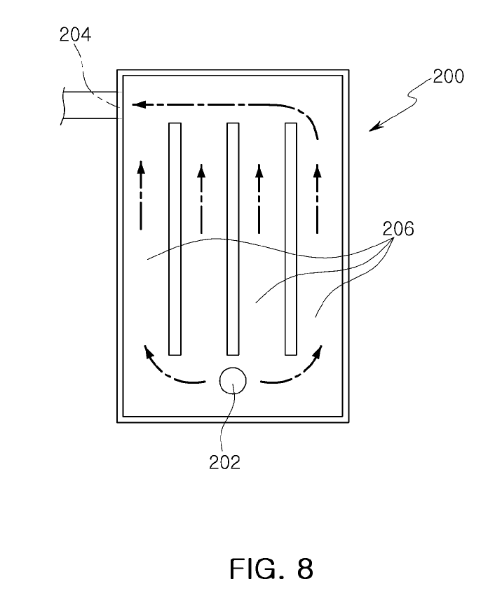

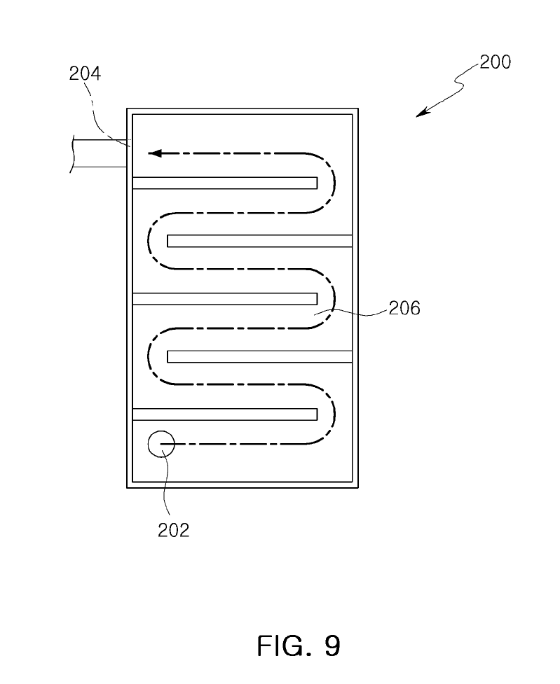

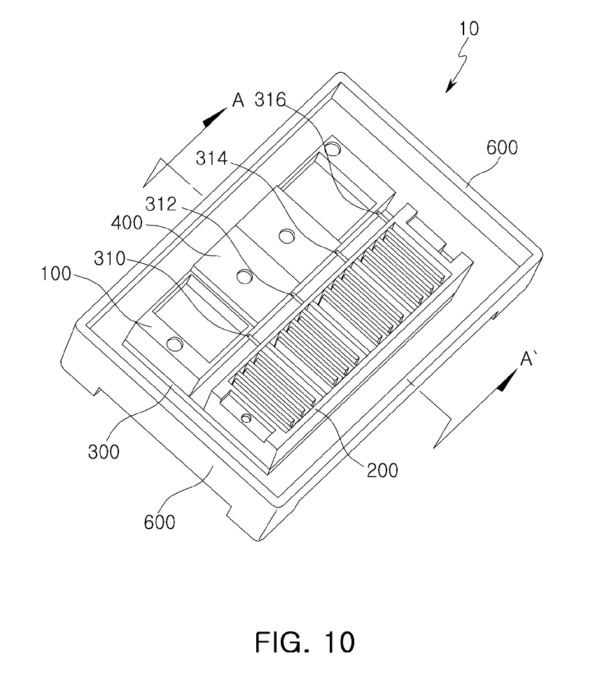

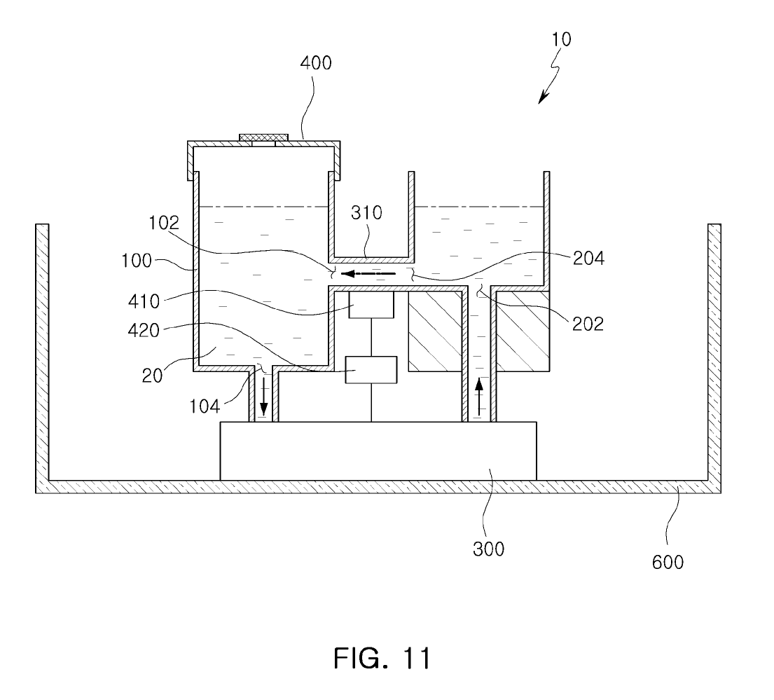

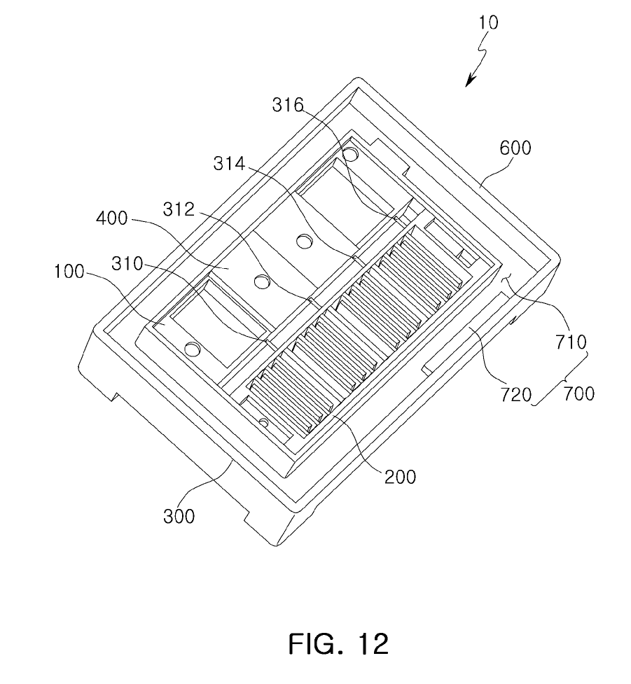

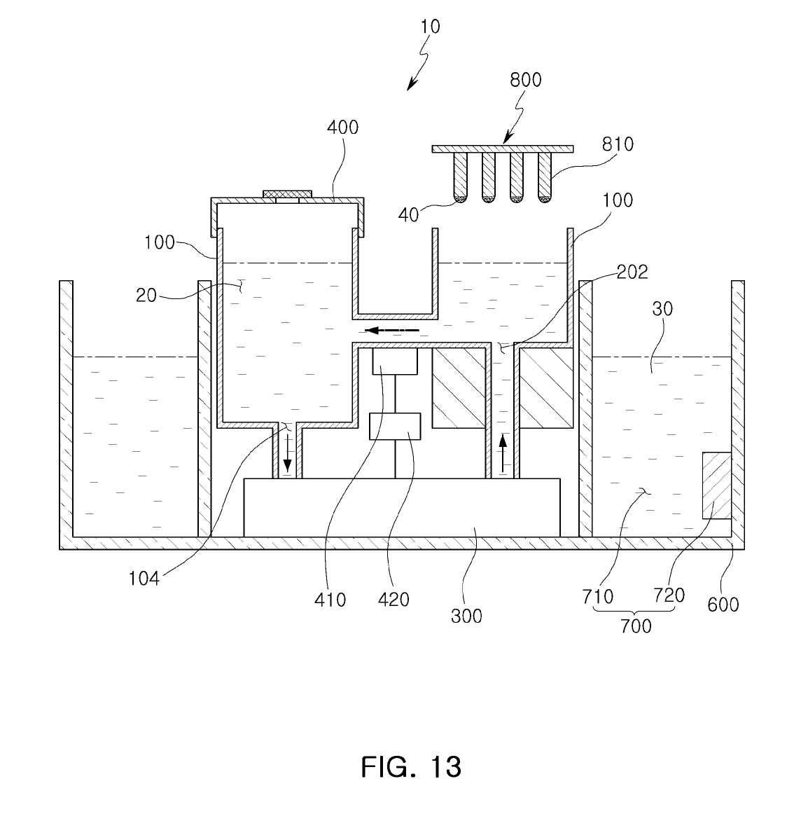

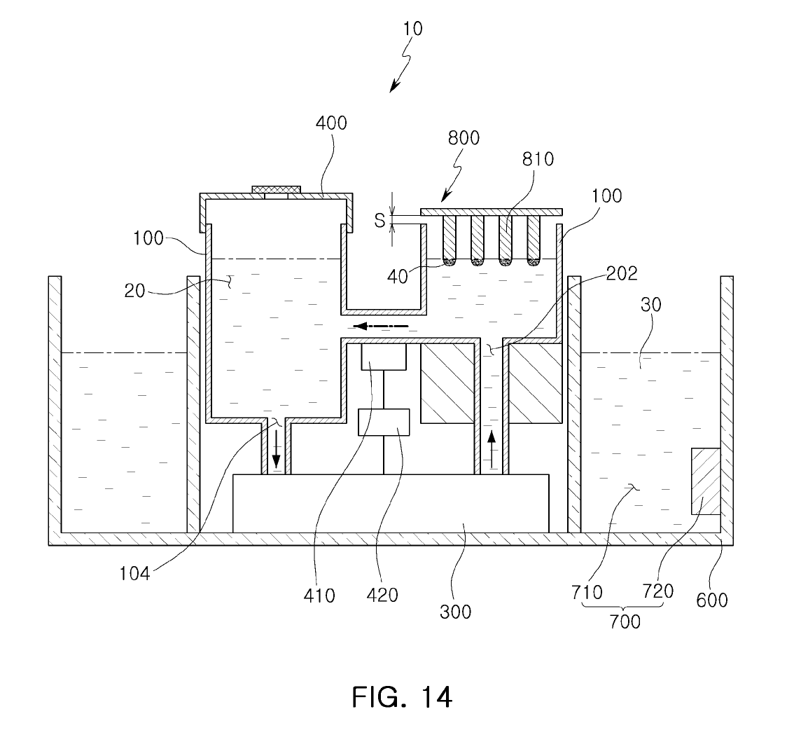

FIG. 1 is a view illustrating a schematic configuration of a cell culture device according to an exemplary embodiment of the present disclosure; FIG. 2 is a view illustrating another form of the cell culture device shown in FIG. 1; FIGS. 3 and 4 are views illustrating an operating state of the cell culture device shown in FIG. 2; FIG. 5 is a view illustrating another form of the cell culture device shown in FIG. 1; FIG. 6 is a plan view illustrating another form of the cell culture device shown in FIG. 1; FIG. 7 is a view illustrating another form of the cell culture device shown in FIG. 1; FIGS. 8 and 9 are views illustrating another form of a cell culture chamber shown in FIG. 1; FIG. 10 is a perspective view of a cell culture device according to another exemplary embodiment of the present disclosure; FIG. 11 is a cross-sectional view of the cell culture device taken along line A-A of FIG. 10; FIG. 12 is a perspective view illustrating another form of the cell culture device shown in FIG. 10; and FIGS. 13 and 14 are cross-sectional views illustrating another form of the cell culture device taken along line A-A of FIG. 10.

A cell culture device according to an exemplary embodiment of the present disclosure will be described with reference to FIG. 1.

The cell culture device 10 according to this exemplary embodiment may include a drug storing chamber 100, a cell culture chamber 200, and a pump unit 300. In addition, the cell culture device 10 may further include an opening and closing unit 400. In addition, the cell culture device 10 may include a drug 20 stored in the drug storing chamber 100. Here, the drug 20 may be interpreted as including several materials required to culture cells. Therefore, in the present description, the drug 20 may also include distilled water in a sterile state, if necessary. In addition, the drug 20 may include components suppressing or promoting cell growth. For example, in the case in which cells to be cultured are cancer cells, the drug 20 may include components suppressing the growth of the cancer cells or killing the cancer cells. In addition, the cell culture device 10 may further include a cell chip 800 (See FIG. 13). Here, the cell chip 800 may have a form in which a single kind of cells or plural kinds of cells are attached thereto. For example, the cell chip 800 may have at least one cell attached onto one surface thereof.

Next, main components of the cell culture device 10 will be described.

The drug storing chamber 100 may have a form in which it may store the drug 20 therein. For example, the drug storing chamber 100 may have a container form in which an inner portion thereof is empty, so that a liquid-state material may be stored therein. In addition, the drug storing chamber 100 may include an inlet 102 and an outlet 104 for circulating the drug stored therein. For example, the drug storing chamber 100 may have the inlet 102 formed at a side thereof and have the outlet 104 formed at a bottom thereof. Here, the inlet 102 may be connected to the cell culture chamber 200 and the outlet 104 may be connected to the pump unit 300. This form may be advantageous for circulating the drug 20 using a hydraulic head.

The drug storing chamber 100 may have an effective volume (volume in which the drug may be substantially stored) larger than that of the cell culture chamber 200. For example, an amount of drug stored in the drug storing chamber 100 may be larger than that of drug stored in the cell culture chamber 200. This may be advantageous for circulating the drug over a long period of time. However, the drug storing chamber 100 and the cell culture chamber 200 may have the same effective volume, if necessary.

The cell culture chamber 200 may have a form in which it may store the drug 20 therein, similar to the drug storing chamber 100. In addition, the cell culture chamber 200 may have a form in which one surface thereof is opened so that the cell chip 800 may be accommodated therein. In addition, the cell culture chamber 200 may include an inlet 202 and an outlet 204 for circulating the drug stored therein. For example, the cell culture chamber 200 may have the outlet 204 formed at a side thereof and have the inlet 202 formed at a bottom thereof. Here, the inlet 202 may be connected to the pump unit 300 and the outlet 204 may be connected to the inlet 102 of the drug storing chamber 100. For reference, a reference numeral 310 may indicate a pipe connecting the outlet 204 and the inlet 102 to each other.

The cell culture chamber 200 may be disposed in parallel with the drug storing chamber 100. For example, the cell culture chamber 200 may be disposed at one side of the drug storing chamber 100, as shown in FIG. 1. In addition, the cell culture chamber 200 may be disposed in a position higher than that of the drug storing chamber 100. For example, a bottom surface of the cell culture chamber 200 may be disposed in a position higher than that of a bottom surface of the drug storing chamber 100 by a first height h. To this end, the cell culture chamber 200 may have a support structure formed therebeneath. In addition, the outlet 204 of the cell culture chamber 200 may be formed at a height that is substantially the same as that of the bottom surface of the cell culture chamber 200. This structure of the cell culture chamber 200 may be advantageous for circulating the drug 20 using a hydraulic head.

The pump unit 300 may circulate the drug 20. For example, the pump unit 300 may generate negative pressure in the drug storing chamber 100 and generate positive pressure in the cell culture chamber 200. As an example, the pump unit 300 may withdraw the drug 20 stored in the drug storing chamber 100 and discharge the withdrawn drug 20 to the cell culture chamber 200. To this end, the pump unit 300 may be connected to both of the drug storing chamber 100 and the cell culture chamber 200.

The pump unit 300 may include a pump. For example, the pump unit 300 may include a pump enabling circulation of the drug 20 and pipes each connecting the pump to the drug storing chamber 100 and the cell culture chamber 200. In addition, the pump unit 300 may further include valves each mounted in the pipes. Here, the valves may be controlled by a control unit 420 (See FIG. 2) to be described below.

The opening and closing unit 400 may be formed on the drug storing chamber 100. For example, the opening and closing unit 400 may cover an opened part of an upper portion of the drug storing chamber 100. In addition, the opening and closing unit 400 may selectively open and close the opened part of the upper portion. For example, the opening and closing unit 400 may close the opened part of the upper portion so that an upper space of the drug storing chamber 100 is completely blocked from the outside. In addition, the opening and closing unit 400 may also open the opened part of the upper portion so that the upper space of the drug storing chamber 100 is connected to the outside.

The opening and closing unit 400 configured as described above may facilitate circulation of the drug 20. For example, the opening and closing unit 400 may form negative pressure in the drug storing chamber 100. As an example, when the drug 20 is discharged through the outlet 104 in the state in which the opened part of the upper portion of the drug storing chamber 100 is closed by the opening and closing unit 400, an inner portion of the drug storing chamber 100 may be maintained at pressure lower than atmospheric pressure. In this case, the drug 20 of the cell culture chamber 200 may naturally move to the drug storing chamber 100 until internal pressure of the drug storing chamber 100 becomes substantially the same as the atmospheric pressure. Therefore, according to this exemplary embodiment, the drug 20 may be smoothly circulated.

Next, another form of the cell culture device according to an exemplary embodiment of the present disclosure will be described with reference to FIGS. 2 through 4.

Another form of the cell culture device 10 may further include a flow rate measuring unit 410 and a control unit 420. For example, the flow rate measuring unit 410 may be mounted on the pipe 310 and the control unit 420 may be mounted on the pump unit 300. However, positions of the flow rate measuring unit 410 and the control unit 420 are not limited to the above-mentioned positions. For example, the flow rate measuring unit 410 and the control unit 420 may also be formed integrally with the pump unit 300.

The flow rate measuring unit 410 may measure a circulation amount of the drug 20. For example, the flow rate measuring unit 410 may measure a flow rate of the drug 20 discharged from the drug storing chamber 100 through the outlet 104 or a flow rate of the drug 20 introduced into the drug storing chamber 100 through the inlet 102. Here, a unit of a flow rate measured by the flow rate measuring unit 410 may be measured in μl/s, μl/min, or the like. In addition, a flow rate value measured by the flow rate measuring unit 410 may be transmitted to the control unit 420.

The control unit 420 may be connected to the flow rate measuring unit 410. In addition, the control unit 420 may be connected to the opening and closing unit 400. In addition, the control unit 420 may control an operation of the opening and closing unit 400 based on the measured flow rate value received from the flow rate measuring unit 410.

For example, the control unit 420 may control the opening and closing unit 400 to close the upper portion of the drug storing chamber 100 in the case in which the measured flow rate value exceeds a preset value (that is, a flow rate of the drug 20 is high) (See FIG. 3). In this case, internal pressure P in the drug storing chamber 100 may be substantially lower than atmospheric pressure Pair. In addition, the control unit 420 may control the opening and closing unit 400 to open the upper portion of the drug storing chamber 100 in the case in which the measured flow rate value is a equal to a preset value or less (that is, a flow rate of the drug 20 is low) (See FIG. 4). In this case, internal pressure P of the drug storing chamber 100 may be substantially the same as atmospheric pressure Pair.

Meanwhile, the case in which the control unit 420 controls the opening and closing unit 400 based on a flow rate of the drug 20 has been described in the present form. However, a reference in controlling the opening and closing unit 400 is not limited to the flow rate of the drug 20. For example, the control unit 420 may also control the opening and closing unit 400 based on viscosity or other characteristics of the drug 20. To this end, the control unit 420 may include a storing part storing a measured value and a preset value, an arithmetic circuit comparing the measured value and the preset value with each other, a logic circuit controlling an operation of the opening and closing unit 400 based on a comparison value, and the like.

Next, another form of the cell culture device according to an exemplary embodiment of the present disclosure will be described with reference to FIGS. 5 through 7.

Another form of the cell culture device 10 may be different in a structure of a pump unit 300 from the above-mentioned forms, as shown in FIG. 5. For example, in this form, the pump unit 300 may be disposed below the cell culture chamber 200. This form may have an advantage that the pump unit 300 may be used as a support structure for maintaining the cell culture chamber 200 at a predetermined height. Therefore, in this form, the support structure for supporting the cell culture chamber 200 may be omitted.

Another form of the cell culture device 10 may include a plurality of cell culture chambers 200: 210, 220, and 230, as shown in FIG. 6. For example, this form of the cell culture device 10 may include three cell culture chambers 200: 210, 220, and 230. In addition, the cell culture device 10 may include drug storing chambers 100 corresponding to the same number as that of cell culture chambers 200: 210, 220, and 230. Here, three cell culture chambers 200: 210, 220, and 230 may accommodate different drugs therein, respectively. Alternatively, three cell culture chambers 200: 210, 220, and 230 may accommodate the same drug therein, respectively. For reference, reference numerals 310, 312, and 314 may indicate pipes each connecting the cell culture chambers 200: 210, 220, and 230 and the drug storing chambers 100 to each other.

Another form of the cell culture device 10 may further include a filtering unit 500, as shown in FIG. 7. For example, the filtering unit 500 may be disposed between the drug storing chamber 100 and the pump unit 300. The filtering unit 500 may filter foreign objects included in the drug 20. For example, the filtering unit 500 may filter a material unnecessary for a cell culture experiment or a material hindering the cell culture experiment and discharge the filtered material to the outside. Alternatively, the filtering unit 500 may filter a material hindering an operation of the pump unit 300 or a material (for example, a lump causing the pipe to be blocked, or the like) hindering circulation of the drug 20. For reference, although the case in which the filtering unit 500 is formed at the outlet 104 of the drug storing chamber 100 has been shown in FIG. 7, a position of the filtering unit 500 may be changed if necessary. For example, the filtering unit 500 may be mounted in the pipe 310.

Next, another form of the cell culture chamber will be described with reference to FIGS. 8 and 9. For reference, a cell culture chamber to be described below may be applied to various forms of cell culture devices including the cell culture devices described above.

The cell culture chamber 200 may include at least one channel 206 for circulating the drug 20. As an example, the cell culture chamber 200 may include a plurality of channels 206, as shown in FIG. 8. The cell culture chamber 200 having the above-mentioned form may be advantageous for an experiment for culturing various cells. As another example, the cell culture chamber 200 may include a channel 206 having a zigzag form, as shown in FIG. 9. The cell culture chamber 200 having the above-mentioned form may allow the drug 20 to stay therein for a long period of time. Therefore, the cell culture chamber 200 having the above-mentioned form may be advantageous in the case in which a sufficient reaction time between the drug 20 and the cells is required.

Next, a cell culture device according to another exemplary embodiment of the present disclosure will be described with reference to FIGS. 10 through 14. For reference, in the following description, the same components as those of the cell culture device according to an exemplary embodiment of the present disclosure described above will be denoted by the same reference numerals and a description thereof will be omitted.

The cell culture device 10 according to this exemplary embodiment may further include a housing 600. For example, the cell culture device 10 may include the housing 600 accommodating all of the drug storing chamber 100, the cell culture chamber 200, and the pump unit 300 therein. Here, the housing 600 may have a form in which an upper portion thereof is opened so as to allow for easy observation of the drug storing chamber 100 and the cell culture chamber 200. However, a form of the housing 600 is not limited to the form shown in FIG. 10. For example, the housing 600 may also accommodate only at least one of the drug storing chamber 100, the cell culture chamber 200, and the pump unit 300 therein. In addition, the housing 600 may have an internal space in which significant portions of the drug storing chamber 100, the cell culture chamber 200, and the pump unit 300 may be accommodated, as shown in FIG. 11. The housing 600 having the above-mentioned shape may be advantageous for preventing external materials from being introduced into the drug storing chamber 100 and the cell culture chamber 200.

The cell culture device may further include a second housing (not shown). For example, the cell culture device 10 may further include the second housing (that is, a cover) covering an upper portion of the housing 600. The cell culture device 10 configured as described above may be easily stored and moved.

Another form of the cell culture device 10 may further include a humidity controlling unit 700, as shown in FIG. 12. For example, the housing 600 may be provided with the humidity controlling unit 700 for maintaining an optimal environment required for culturing the cells.

The humidity controlling unit 700 may constantly maintain ambient humidity in the cell culture chamber 200. For example, the humidity controlling unit 700 may include a water storing chamber 710 and a heater 720. Here, the water storing chamber 710 may provide water required for maintaining humidity and the heater 720 may provide a temperature condition for evaporating the water 30 stored in the water storing chamber 710. In addition, the humidity controlling unit 700 may also control a temperature of the water 30 stored in the water storing chamber 720 to control an ambient temperature of the cell culture chamber 200.

Another form of the cell culture device 10 may further include a cell chip 800, as shown in FIGS. 13 and 14. For example, the cell culture chamber 200 may be provided with the cell chip 800. In addition, the cell chip 800 may have a form advantageous for culturing the cells. For example, the cell chip 800 may have a plurality of protrusions 810 so that the cells 40 and the drug 20 may easily contact each other. In addition, the cell chip 800 may be maintained at a predetermined height from an upper surface of the cell culture chamber 200. For example, the cell chip 800 and a wall surface of the cell culture chamber 200 may have a predetermined space S formed therebetween, as shown in FIG. 14. This form may facilitate the introduction of fresh air and moisture into the cell culture chamber 200.

As set forth above, the cell culture device according to an exemplary embodiment of the present disclosure may be advantageous for constantly and accurately circulating a small amount of drug.

While exemplary embodiments have been shown and described above, it will be apparent to those skilled in the art that modifications and variations could be made without departing from the spirit and scope of the present disclosure as defined by the appended claims.

What is claimed is:

1. A cell culture device comprising:
a drug storing chamber;
a cell culture chamber connected to the drug storing chamber and disposed in a position higher than that of the drug storing chamber;
a pump unit circulating a drug from the drug storing chamber to the cell culture chamber; and
an opening and closing unit closing an opened part of the drug storing chamber so that the drug storing chamber is isolated from ambient atmosphere.

2. The cell culture device of claim 1, further comprising a flow rate measuring unit measuring a flow rate of the drug circulated from the drug storing chamber to the cell culture chamber.

3. The cell culture device of claim 2, further comprising a control unit comparing a measured signal received from the flow rate measuring unit with a preset value and controlling an operation of the opening and closing unit.

4. The cell culture device of claim 3, wherein the control unit includes a logic circuit operating the opening and closing unit so as to open the opened part when the measured signal is equal to the preset value or less and operating the opening and closing unit so as to close the opened part when the measured signal exceeds the preset value.

5. The cell culture device of claim 1, wherein the pump unit is disposed below the drug storing chamber.

6. The cell culture device of claim 1, wherein the drug storing chamber is provided in plural.

7. The cell culture device of claim 1, wherein the cell culture chamber is provided in plural.

8. The cell culture device of claim 1, wherein the cell culture chamber includes at least one channel.

9. The cell culture device of claim 8, wherein the at least one channel has a zigzag form.

10. The cell culture device of claim 1, further comprising a filtering unit filtering foreign objects from the drug circulated from the drug storing chamber to the cell culture chamber.

11. A cell culture device comprising:
a housing having a drug storing chamber and a cell culture chamber formed therein;
a pump unit circulating a drug from the drug storing chamber to the cell culture chamber; and
an opening and closing unit closing an opened part of the drug storing chamber so that the drug storing chamber is isolated from ambient atmosphere.

12. The cell culture device of claim 11, further comprising a flow rate measuring unit measuring a flow rate of the drug circulated from the drug storing chamber to the cell culture chamber.

13. The cell culture device of claim 12, further comprising a control unit comparing a measured signal received from the flow rate measuring unit with a preset value and controlling an operation of the opening and closing unit.

14. The cell culture device of claim 13, wherein the control unit includes a logic circuit operating the opening and closing unit so as to open the opened part when the measured signal is equal to the preset value or less and operating the opening and closing unit so as to close the opened part when the measured signal exceeds the preset value.

15. The cell culture device of claim 11, wherein the pump unit is disposed below the drug storing chamber.

16. The cell culture device of claim 11, wherein the drug storing chamber is provided in plural.

17. The cell culture device of claim 11, wherein the cell culture chamber is provided in plural.

18. The cell culture device of claim 11, wherein the cell culture chamber includes at least one channel.

19. The cell culture device of claim 18, wherein the at least one channel has a zigzag form.

20. The cell culture device of claim 11, further comprising a filtering unit filtering foreign objects from the drug circulated from the drug storing chamber to the cell culture chamber.

21. The cell culture device of claim 11, further comprising a humidity controlling unit formed in the housing so as to constantly maintain humidity within the cell culture chamber.

22. The cell culture device of claim 21, wherein the humidity controlling unit includes:
a water storing chamber formed in the housing; and
a heater evaporating water stored in the water storing chamber.

23. The cell culture device of claim 11, further comprising a cell chip mounted on the cell culture chamber.

24. The cell culture device of claim 23, wherein the cell chip includes a plurality of protrusions extended into the cell culture chamber and having cell materials fixed thereto, respectively.
